(19) United States
(12) Patent Application Publication
(10) Pub. No.: US 2017/0106370 A1
(43) Pub. Date: Apr. 20, 2017

(54) AMPLIFICATION APPARATUS, AMPLIFICATION METHOD AND AMPLIFICATION SYSTEM

(71) Applicant: NEC Corporation, Minato-ku, Tokyo (JP)

(72) Inventors: Minoru ASOGAWA, Tokyo (JP); Yoshinori MISHINA, Tokyo (JP); Yasuo IIMURA, Tokyo (JP); Hisashi HAGIWARA, Tokyo (JP); Ryou YAMAZAKI, Tokyo (JP)

(73) Assignee: NEC Corporation, Minato-ku, Tokyo (JP)

(21) Appl. No.: 15/129,276

(22) PCT Filed: Mar. 31, 2014
(86) PCT No.: PCT/JP2014/059560

§ 371 Date: Sep. 26, 2016

(51) Int. Cl. B01L 7/00; B01L 3/00; C12Q 1/68

(52) U.S. Cl. CPC B01L 7/52; C12Q 1/6844; B01L 3/502707; B01L 2300/0816; B01L 2300/0864; B01L 2300/0887; B01L 2300/123; B01L 2300/087; B01L 2300/1822

(57) ABSTRACT

An amplification apparatus comprises an amplification unit, monitoring unit and control unit. The amplification unit is a means for amplifying desired nucleic acid sequence by heating and cooling sample solution. The monitoring unit is a means for monitoring amount of amplicon as nucleic acid sequence amplified by the amplification unit. The control unit is a means for terminating amplification process by the amplification unit based on the amount of amplicon monitored by the monitoring unit.

This application is a National Stage Entry of PCT/JP2014/059560 filed on Mar. 31, 2014, the content of which is incorporated herein by reference, in its entirety.

### TECHNICAL FIELD

The present invention relates to an amplification apparatus, amplification method and amplification system. Particularly, the present invention relates to an amplification apparatus, amplification method and amplification system which amplifies desired nucleic acid sequence.

### BACKGROUND

PCR (Polymerase Chain Reaction) is a reaction carried out in genetic engineering field etc., in which a desired nucleic acid sequence is amplified in order to synthesize amplicon. In addition, an apparatus for carrying out PCR, that is a thermal cycler, has been developed (see, for example, Patent Literature 1).

### Patent Literature 1

Japanese Patent Kohyo Publication: No. 2008-519600A

### SUMMARY

In PCR protocol carried out without adjustment in amount of template DNA (Deoxyribonucleic Acid), there are cases where excessive amplicon is synthesized due to too many cycle numbers, and on the other hand, insufficient amplicon is synthesized due to too few cycle numbers. In such cases, there is a case where desired result would not be obtained. For example, in DNA test utilizing microsatellites, repeat numbers in repeat sequence is measured based on length (bases) of the amplicon. Herein, if excessive amplicon is synthesized, DNA band would have smear form, resulting in that length of the amplicon cannot be measure accurately. In addition, in a condition where amplicon amount is insufficient, detection peak of the DNA band would be too weak and buried in noise, resulting in that length of the amplicon cannot be measured.

Although amplicon may be synthesized to a prospected amplicon amount with a PCR protocol in which amount of template DNA is adjusted, it imposes large burden into an operator since it requires labor for measuring amount of template DNA. In addition, in a case where DNA left at a scene of a crime is applied to a test and the like, the amount of available DNA is restricted, thus there is also a case where amount of template DNA is impossible to be adjusted.

It is a purpose of the present invention to provide an amplification apparatus, amplification method and amplification system contributing to synthesis of desired nucleic acid sequence at a suitable amount.

According to first aspect of the present invention, there is provided an amplification apparatus comprising: an amplification unit amplifying desired sequence by heating and cooling sample solution; a monitoring unit monitoring amount of amplicon as nucleic acid sequence amplified by the amplification unit; and a control unit terminating amplification process by the amplification unit based on the amount of amplicon monitored by the monitoring unit.

According to second aspect of the present invention, there is provided an amplification method comprising: amplifying desired nucleic acid sequence by heating and cooling sample solution; measuring the amount of amplicon as amplified nucleic acid sequence; terminating amplification of the desired nucleic acid sequence based on the measured amplicon amount.

According to third aspect of the present invention, there is provided an amplification system comprising: a microchip which comprises a plurality of laminated elastic sheets and in which amplification chambers for amplifying desired nucleic acid sequences are constructed at inadhesive site between the elastic sheets; and an amplification apparatus comprising: an amplification unit amplifying desired nucleic acid sequence by heating and cooling sample solutions in the amplification chambers; a monitoring unit monitoring amount of amplicon in the amplification chambers; and a control unit terminating amplification process by the amplification unit based on the amount of amplicon monitored by the monitoring unit.

According to each aspect of the present invention, there are provided an amplification apparatus, amplification method and amplification system contributing to synthesis of desired nucleic acid sequence at a suitable amount.

### BRIEF DESCRIPTION OF THE DRAWINGS

FIG. 1 is an explanatory view of a construction of an exemplary amplification apparatus.

FIG. 2 is an explanatory view of operation in an exemplary amplification apparatus.

FIG. 3 is a perspective view showing an example of entire construction of a microchip controlling apparatus of a first embodiment.

FIG. 4 is a schematic view showing an exemplary construction of a microchip of the first embodiment.

FIG. 5 is a schematic plan view showing an exemplary DNA extraction/PCR section of the first embodiment.

FIG. 6 is a diagram showing an exemplary schematic sectional view of the microchip of the first embodiment.

FIG. 7 is an explanatory view of flow path opening/closing mechanism and liquid transferring mechanism by the microchip controlling apparatus.

FIG. 8 is a sectional view showing an exemplary PCR section, temperature control unit and amplicon amount monitoring unit of the first embodiment.

FIG. 9 is a sectional view showing another exemplary PCR section, temperature control unit and amplicon amount monitoring unit of the first embodiment.

FIG. 10 is a sectional view showing another exemplary PCR section, temperature control unit and amplicon amount monitoring unit of the first embodiment.

FIG. 11 is a sectional view showing another exemplary PCR section, temperature control unit and amplicon amount monitoring unit of the first embodiment.

FIG. 12 is a sectional view showing another exemplary PCR section, temperature control unit and amplicon amount monitoring unit of the first embodiment.

FIG. 13 is a sectional view showing another exemplary PCR section, temperature control unit and amplicon amount monitoring unit of the first embodiment.

FIG. 14 is a flowchart showing an example of PCR step by the controller of the first embodiment.

FIG. 15 is a schematic plan view showing an exemplary electrophoresis section of the first embodiment.

FIG. 16 is a flowchart showing an example of DNA analysis process by the microchip controlling apparatus of the first embodiment.

FIG. 17 is a perspective view showing an exemplary entire construction of the microchip controlling apparatus of the second embodiment.

FIG. 18 is a perspective view showing an exemplary entire construction of the microchip controlling apparatus of the third embodiment.

FIG. 19 is a schematic plan view showing an exemplary final reaction section of the third embodiment.

### PREFERRED MODES

Preferable embodiments will be explained in detail below with reference to drawings. It should be noted that it is not intended to limit the discloser of the present application to the mode shown in the drawings. In addition, symbols are merely attached for convenience in understanding the explanation.

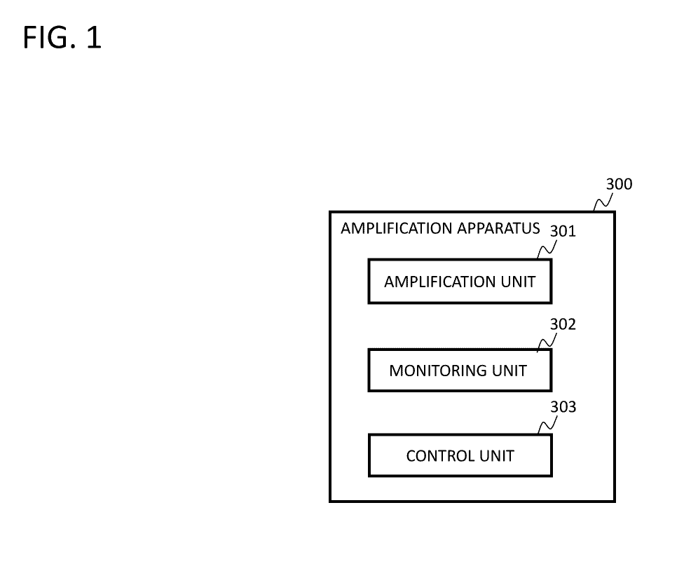

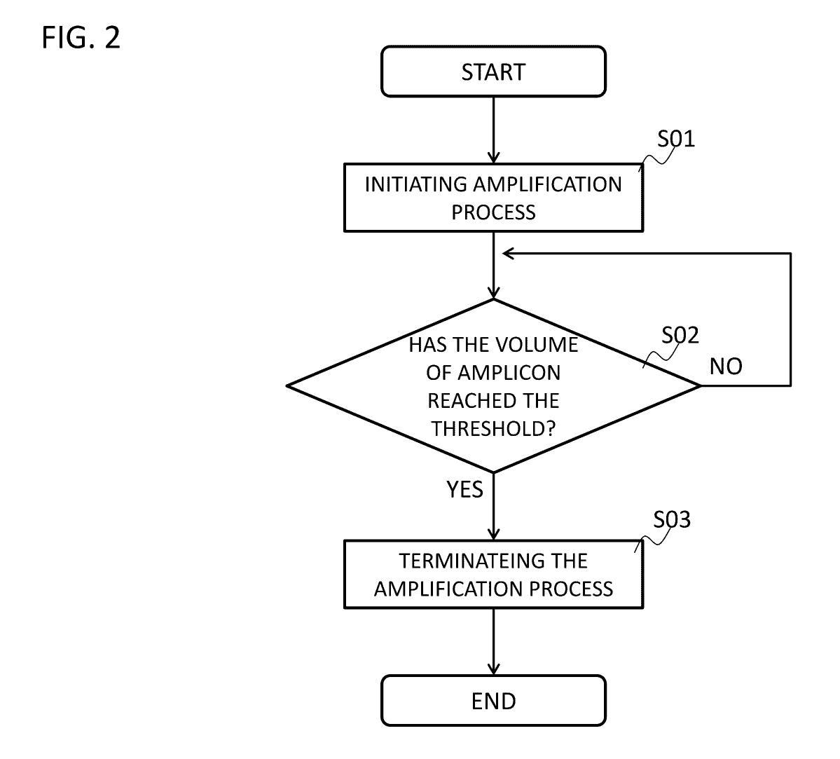

In first, construction and operation of an amplification apparatus of an exemplary embodiment will be explained with reference to FIGS. 1 and 2. When see FIG. 1, an amplification apparatus 300 comprises an amplification unit 301, monitoring unit 302 and control unit 303.

The amplification apparatus 300, as shown in FIG. 2, initiates amplification process in the amplification unit 301 (step S01), and monitors the amount of amplicon as amplified nucleic acid sequence in the monitoring unit 302 (step S02). Under control by the control unit 303, the amplification apparatus 300 terminates the amplification process by the amplification unit 301 based on the amount of amplicon monitored by the monitoring unit 302. Specifically, the amplification apparatus 300 continues the amplification process until the amount of amplicon has reached a preset threshold (step S02, branching to NO). On the other hand, when the amount of amplicon has reached the preset threshold (step S02, branching to YES), the amplification apparatus 300 terminates the amplification process (step S03).

Accordingly, the amplification apparatus 300 of the exemplary embodiment may synthesize amplicon to a suitable amount, but not synthesizing amplicon excessively, with sample solution in which amount of template DNA has not been adjusted. That is, the amplification apparatus 300 may synthesize amplicon at a suitable amount.

### First Embodiment

A specific example of an amplification apparatus and amplification method will be exemplified and explained below with reference to drawings. In a first embodiment, an amplification system will be explained, in which an amplification apparatus disclosed in the present application is applied to a microchip 200 and a microchip controlling apparatus 10 for carrying out PCR. Herein, the microchip controlling apparatus 10 is an apparatus carrying out PCR and electrophoresis for DNA test utilizing microsatellites, in which repeat number in nucleic acid sequence is measured based on length (bases) of the amplicon measured by the microchip controlling apparatus 10.

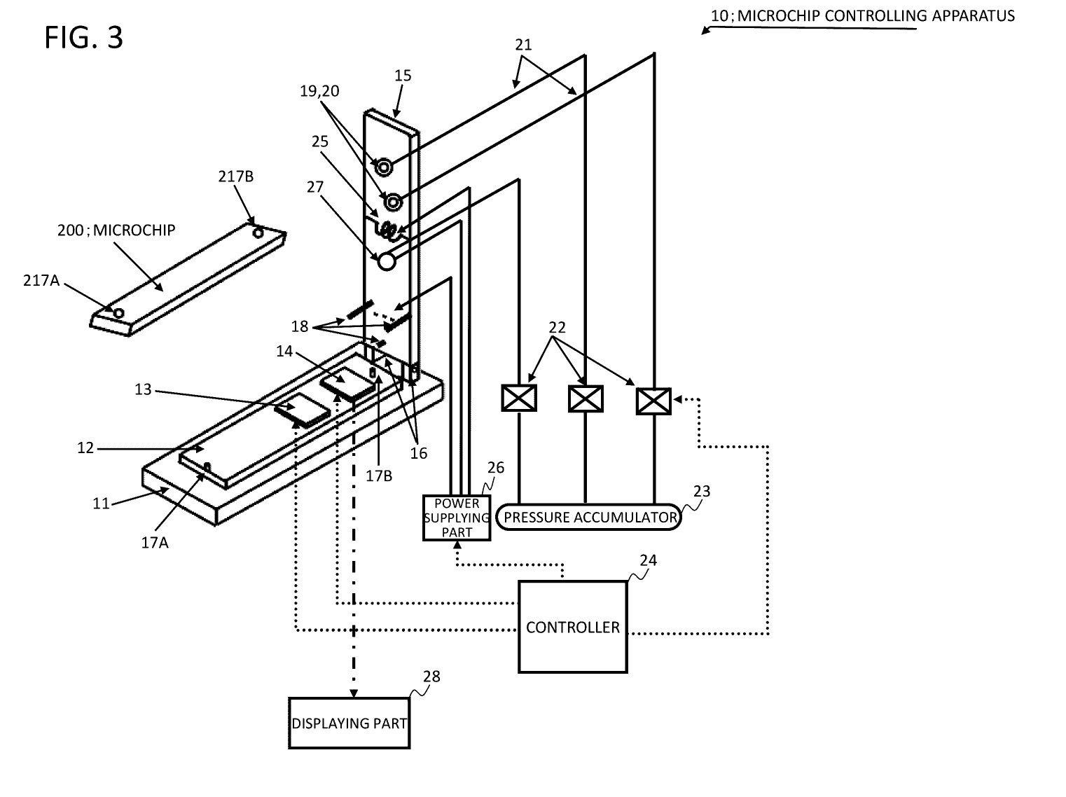

As shown in FIG. 3, in the microchip controlling apparatus 10, a table 12 is arranged on a base station 11, and a temperature control unit 13 (also referred to as an amplification unit) and an electrophoresis unit 14 are arranged on the table 12. In addition the base station 11 and a lid 15 are jointed with a hinge 16 so that the lid 15 may be opened and closed.

The microchip 200 is placed on a predetermined position on the table 12 by engaging pin 17A and pin 17B arranged on the table 12 with pin holes 217A and 217B arranged on the microchip 200. When the lid 15 is closed in a condition where the microchip 200 has been placed on the table 12, a part of region on the microchip 200 where PCR is carried out contacts to the temperature control unit 13. In addition, by closing the lid 15, a region on the microchip 200 where electrophoresis is carried out contacts to the electrophoresis unit 14 and electrodes 18 are inserted into electrode chambers on the microchip 200 via electrode holes arranged on the microchip 200. Herein, detail of the region on the microchip 200 where PCR is carried out and the region where electrophoresis is carried out are explained below.

A plurality of pressurizing holes 19 are arranged on the lid 15. Regions on the lid 15 corresponding to these pressurizing holes 19 are perforated, and the pressurizing holes 19 are communicated to a solenoid valve 22 via tubes 21. In addition, by closing the lid 15, the pressurizing holes 19 and a variety of control holes on the microchip 200 are connected. Herein, it is preferable that the pressurizing holes 19 and the control holes are brought into contact with an interposed sealing mechanism, such as O-rings 20. The variety of control holes on the microchip 200 will be explained below.

A pressure accumulator 23 stores pressurizing medium, such as compressed air, and a controller 24 controls a solenoid valve 22 so that pressurizing medium is injected into or ejected from the control holes on the microchip 200 via the pressurizing holes 19. Herein, internal pressure in the pressure accumulator 23 is controlled by a pressure sensor, pump etc., not shown, so as to be maintained at a predetermined pressure.

A DNA extracting unit 25 is arranged on the lid 15, which extracts sample DNA or template DNA from sample solution. In a case where the DNA extracting unit 25 extracts sample DNA with, for example, magnetic beads (silica), the DNA extracting unit 25 comprises neodymium magnets to which magnetic beads are attached. Under control by the controller 24, the DNA extracting unit 25 moves the neodymium magnets to the DNA extracting section 244 or moves the neodymium magnet away from the DNA extracting section 244.

An amplicon amount monitoring unit 27 is also arranged on the lid 15. The construction and function of the amplicon amount monitoring unit 27 will be explained below.

The temperature control unit 13 has temperature controlling mechanism for carrying out PCR and denaturation process. Specifically, the temperature control unit 13 comprises a temperature sensor, heat conductor, Peltier element (thermoelectric element), heat releasing plate etc., which acquires temperature at the region where PCR is carried out from the temperature sensor and controlling heating or cooling on the Peltier element based on the acquired temperature to achieve temperature control at the region where PCR is carried out.

An electrophoresis unit 14 is a mechanism carrying out capillary electrophoresis and detection of fluorescent label, which comprises an excitation device, such as a halogen lamp, mercury lamp and laser beam, as well as a filter and camera. When capillary electrophoresis is initiated by applying DC voltage to the electrodes 18 via a power supplying part 26, the electrophoresis unit 14 monitors fluorescent label flowing in capillary and outputs detection result in which change in fluorescence intensity is graphed in a time dependent manner via a displaying part 28.

Herein, the controller 24 may be realized with a computer program which makes hardware as a computer installed in the microchip controlling apparatus 10 to execute a process by the controller 24 as described below.

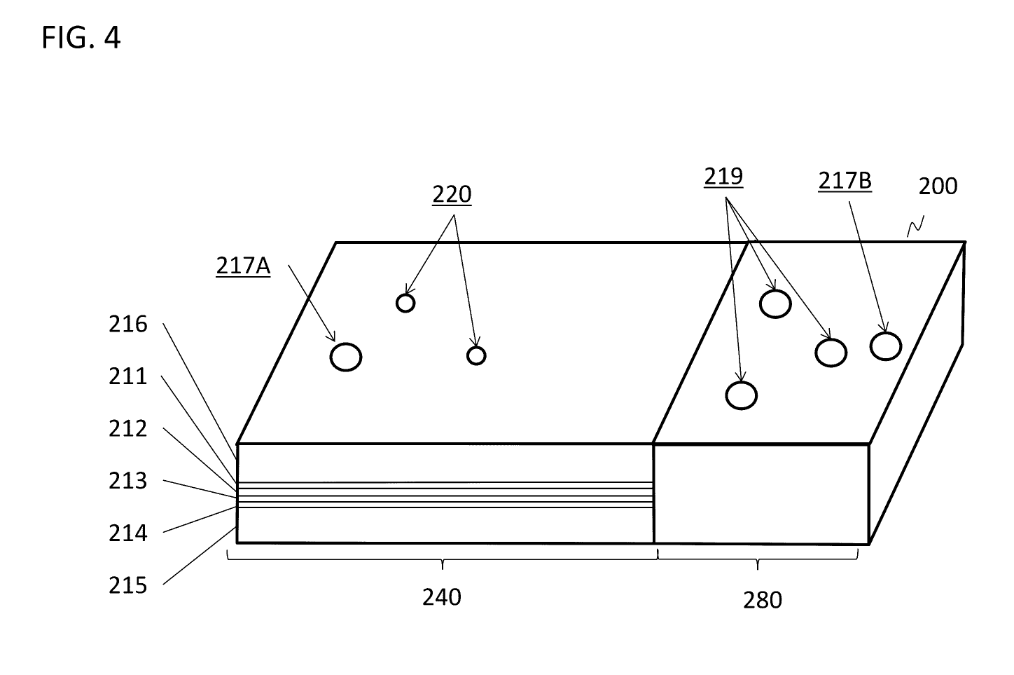

As shown in FIG. 4, the microchip 200 comprises a DNA extraction/PCR section 240 and electrophoresis section 280. The DNA extraction/PCR section 240 is consisting of fourth elastic sheet 214 superposed on a resin plate 215, third elastic sheet 213 superposed on the fourth elastic sheet 214, second elastic sheet 212 superposed on the third elastic sheet 213, first elastic sheet 211 superposed on the second elastic sheet 212, and a resin plate 216 superposed on the first elastic sheet 211. The elastic sheets 211 to 214 are adhered each other partial exceptions. Inadhesive site may be expanded by injection of medium, such as liquid and air, and then middle layer is formed between the elastic sheets 211 to 214. Herein, a middle layer between the first elastic sheet 211 and the second elastic sheet 212 is referred to as first middle layer, a middle layer between the second elastic sheet 212 and third elastic sheet 213 is referred to as second middle layer, and a middle layer between the third elastic sheet 213 and the fourth elastic sheet 214 is referred to as third middle layer.

It is preferable that the elastic sheets 211 to 214 have elasticity, heat resistance, and acid/alkali resistance. It is preferable that the resin plates 215, 216 have hardness to an extent such that they may control extension of the elastic sheets 211 to 214. Herein, the resin plate 215 may be also arranged on the base station 11 of the microchip controlling apparatus 10. A variety of control holes, such as a pin hole 217A and medium injecting/ejecting hole 220, are formed on the DNA extraction/PCR section 240. In addition, a variety of control holes, such as pin hole 217B and electrode holes 219, are formed on the electrophoresis section 280. Note that FIG. 4 is partially simplified for clarity.

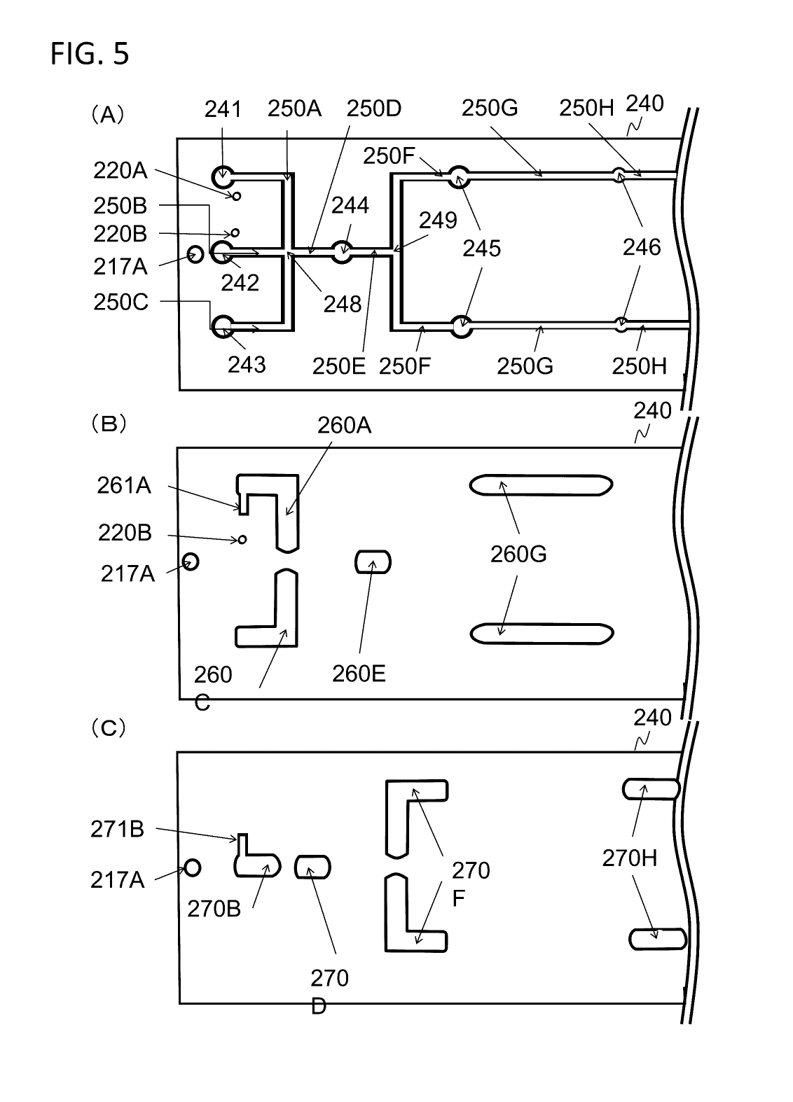

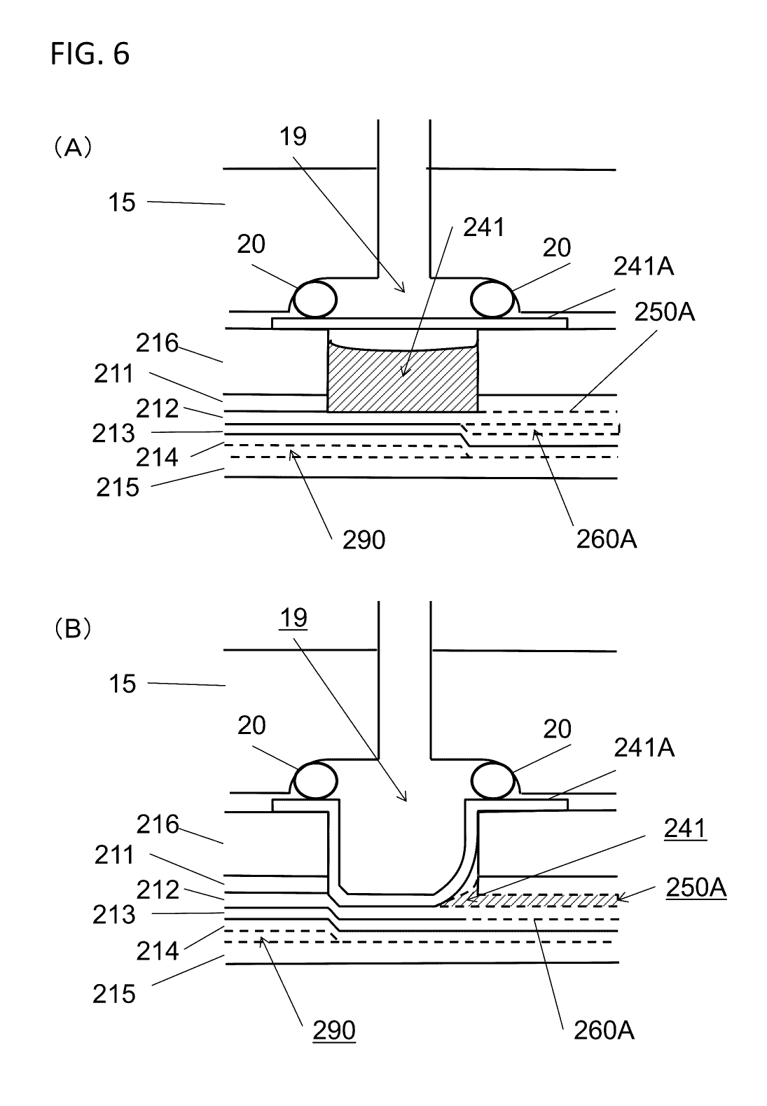

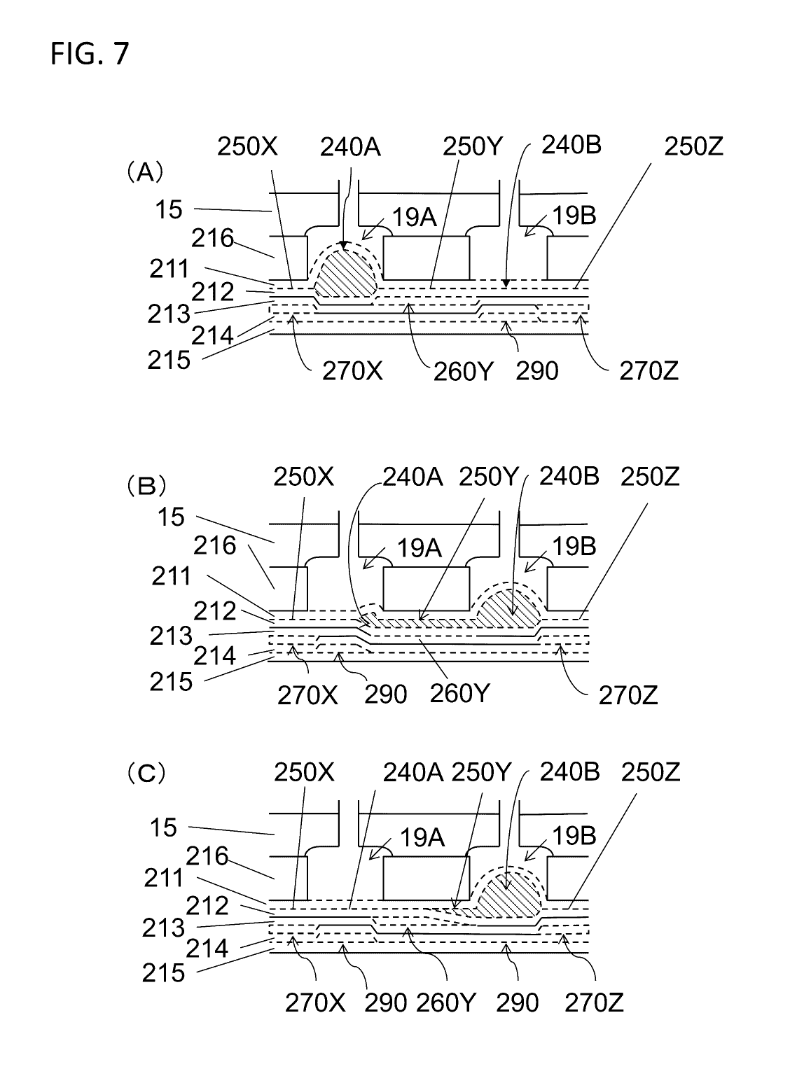

FIG. 5 is a schematic plan view showing an example of the DNA extraction/PCR section 240. As shown in FIG. 5(A), the DNA extraction/PCR section 240 comprises a sample solution injection section 241, wash buffer injection section 242 and elution buffer injection section 243, in which DNA extracting section 244, PCR section 245 and volume determination section 246 are formed as first middle layer. Herein, the PCR section 245 is also referred to as the amplification chamber. The sample solution injection section 241 is connected with flow path 250A. The wash buffer injection section 242 is connected with flow path 250B. The elution buffer injection section 243 is connected with flow path 250C. The flow paths 250A to C flow together into a confluence point 248 and being communicated to the DNA extracting section 244 via flow path 250D. In addition, the DNA extracting section 244 is also connected to flow path 250E. The flow path 250E branches at a branching point 249 into a plurality of reaction paths (sample solution is divided) and being communicated with a plurality of PCR sections 245 as flow path 250F. Each PCR section 245 is respectively communicated with a corresponding volume determination section 246 via a flow path 250G. Herein, the flow paths 250A to H and the like are inadhesive site between the first elastic sheet 211 and the second elastic sheet 212, which are formed by injection of liquid etc. thereinto. That is, space section 290 is arranged between the fourth elastic sheet 214 and the resin plate 215. Upon formation of the flow path 250 and the like, the elastic sheet 214 is pressed down into the space section 290 (see FIG. 6 and FIG. 7). In addition, in the present application, the reaction path means a single flow path from the flow path 250F through the PCR section 245 to the sample flow path 281. In other words, “each of reaction paths” and “each of PCR section 245” are interpreted interchangeably.

As shown in FIG. 5(B), flow path opening/closing sections 260A, C, E, G corresponding to the flow paths 250A, C, E, G are formed on the DNA extraction/PCR section 240 as second middle layer between the second elastic sheet 212 and the third elastic sheet 213. In addition, as shown in FIG. 5(C), flow path opening/closing sections 270B, D, F, H corresponding to the flow paths 250B, D, F, H are formed as third middle layer between the third elastic sheet 213 and the fourth elastic sheet 214.

As shown in FIG. 5(B), the flow path opening/closing section 260A comprises medium flow path 261A at upstream side of the flow path 250A (that is, the side of the sample solution injection section 241) and being connected to a pressurizing hole 19 arranged on the lid 15 via a medium injecting/ejecting hole 220A through the first middle layer, first elastic sheet 211 and resin plate 216. In addition, as shown in FIG. 5(C), the flow path opening/closing section 270B comprises medium flow path 271B at upstream side of the flow path 250B (that is, the side of the wash buffer injection section 242) and being connected to a pressurizing hole 19 arranged on the lid 15 via a medium injecting/ejecting hole 220B through the second middle layer, second elastic sheet 212, first middle layer, first elastic sheet 211 and resin plate 216. Herein, only the medium flow path 261A, medium injecting/ejecting hole 220A, medium flow path 271B and medium injecting/ejecting hole 220B are shown in FIG. 5, and the other constructions are omitted.

As shown in FIG. 6, the sample solution injection section 241 is a through hole perforating the resin plate 216 and the first elastic sheet 211, into which sample solution is injected by an operator (manual operation or automatic injection unit) and which is covered with a cover film 241A. The sample solution is solution in which cells obtained from a subject are suspended into lysis buffer (for example, SDS/LiOAc solution (sodium dodecyl sulfate/lithium acetate solution)). Specifically, the sample solution injection section 241 is connected to a pressurizing hole 19 arranged on the lid 15 via the cover film 241A and O-ring 20. Hereinafter, the pressurizing hole 19 is interpreted to comprise the O-ring 20, and explanation for the O-ring 20 would be omitted.

Next, a case is considered with reference to FIG. 5, where sample solution is transferred from the sample solution injection section 241 to the DNA extracting section 244 through the flow paths 250A, D. In first, the microchip controlling apparatus 10 injects pressurizing medium into the flow path opening/closing sections 260C, E and flow path opening/closing sections 270B so as to close flow paths 250B, C, E. Then flow paths 250A, D are opened by releasing pressurizing medium from the flow path opening/closing section 260A and flow path opening/closing section 270D. Then, as shown in FIG. 6(B), the microchip controlling apparatus 10 applies pressurizing medium to the sample solution injection section 241 and presses down the cover film 241A so that the sample solution is extruded to the flow path 250A.

The wash buffer injection section 242 comprises similar construction with the sample solution injection section 241 excepting for that the flow path opening/closing section 270B corresponding to the flow path 250B is arranged as the third middle layer, into which wash buffer is injected by an operator. The wash buffer is, for example, Tris (tris (hydroxymethyl) aminomethane) buffer.

The elution buffer injection section 243 comprises similar construction with the sample solution injection section 241, into which elution buffer is injected by an operator. The elution buffer is buffer for elution of DNA from the DNA extracting section 244 (specifically, magnetic beads) and further comprises polymerase for primer extension reaction, dNTP mix (mixture of deoxyribonucleotide triphosphates), fluorescent substance for measuring the amount of amplicon. Herein, the fluorescent substance comprises, for example, intercalator emitting fluorescence when it is intercalated into double-strand DNA (so-called intercalator method). In addition, the fluorescent substance may be oligo nucleotide probe (so-called TaqMan probe method) in which 5′ terminal is modified with a fluorescent substance and 3′ terminal is modified with a quencher substance. In addition, chimeric probe may be utilized as fluorescent substance, which is consisting of RNA and DNA, in which 5′ terminal is modified with a fluorescent substance and 3′ terminal is modified with a quencher substance (so-called cycling probe method). Herein, in a case where the cycling probe method is used, the elution buffer further comprises RNaseH (ribonuclease H).

Herein, flow path opening/closing mechanism and liquid transferring mechanism by the microchip controlling apparatus 10 will be explained. When liquid flows through the first flow path, the microchip controlling apparatus 10 opens the first flow path by releasing medium from the first flow path opening/closing section so as to contract the first flow path opening/closing section, and then closes the second flow path by injecting medium into the second flow path opening/closing section so as to expand the second flow path opening/closing section. As a specific example, liquid transferring mechanism in the microchip 200 will be explained with reference to FIG. 7, in which liquid in a liquid chamber 240A is transferred to a liquid chamber 240B through a flow path 250Y. The liquid chamber 240A is formed between the first elastic sheet 211 and the second elastic sheet 212 and being connected to flow paths 250X and 250Y. A part corresponding to the liquid chamber 240A on the resin plate 216 is perforated to form a control hole, and pressurizing medium may be injected into/ejected from upper section of the liquid chamber 240A through a pressurizing hole 19A arranged on the lid 15. Similarly, the liquid chamber 240B is connected to the flow paths 250Y and 250Z, and pressurizing medium may be injected into/ejected from upper section of the liquid chamber 240B. The flow paths 250X, Y are closed.

Under such premise, in first, as shown in FIG. 7(A), the microchip controlling apparatus 10 injects pressurizing medium into the flow path opening/closing section 270Z so as to close the flow path 250Z and then releases pressurizing medium from the flow path opening/closing section 260Y so as to open the flow path 250Y. Then, the microchip controlling apparatus 10 applies the pressurizing medium to the liquid chamber 240A through the pressurizing holes 19A. As a result, as shown in FIG. 7(B), liquid extruded from the liquid chamber 240A reaches the liquid chamber 240B through the flow path 250Y, pushes up the first elastic sheet 211 and accumulates in the liquid chamber 240B. When the microchip controlling apparatus 10 determines that impressed pressure of pressurizing medium onto the liquid chamber 240A exceeds a predetermined value and liquid has been ejected from the liquid chamber 240A, the microchip controlling apparatus 10, as shown in FIG. 7(C), injects pressurizing medium into the flow path opening/closing section 260Y from upstream side of the flow path 250Y (that is, the side of the liquid chamber 240A). As a result, liquid in the flow path 250Y is extruded into the liquid chamber 240B and the liquid transfer is completed. After that, since there is no need to close the flow path 250X, the microchip controlling apparatus 10 releases the pressurizing medium from the flow path opening/closing section 270X.

Returning to explanation of FIG. 5, the DNA extracting section 244 is a mechanism arranged for extracting DNA from sample solution. For example, magnetic beads (silica) have been previously stored in the DNA extracting section 244 and sample DNA is extracted from sample solution according to control by the controller 24 and DNA extracting unit 25.

DNA extraction process will be concretely explained. The microchip controlling apparatus 10 comprises neodymium magnets as the DNA extracting unit 25 and magnetic beads coated with silica has been previously stored in the DNA extracting section 244. The microchip controlling apparatus 10 transfers sample solution injected into the sample solution injection section 241 to the DNA extracting section 244 so that DNA is attached on the magnetic beads (silica) stored in the DNA extracting section 244. Then, the magnetic beads are washed with wash buffer stored in the wash buffer injection section 242 so as to extract DNA. Herein, when the microchip controlling apparatus 10 discharges sample solution and wash buffer via a drainage port (not shown), magnetic beads are attached onto the neodymium magnet so that it is prevent that the magnetic beads are discharged together with the sample solution and wash buffer.

DNA extraction method may be modified with reference to a standard protocol etc., for example, rounds of washing may be increased. In addition, the DNA extraction method should not be limited to the method utilizing the magnetic beads, for example, a method utilizing column may be adopted.

The PCR section 245 receives temperature control by the temperature control unit 13 for carrying out PCR. Specifically, primer sets have been previously stored in the PCR section 245, desired nucleic acid sequence in sample DNA (template DNA) extracted in the DNA extracting section 244 is amplified by activity of polymerase contained in the elution buffer. At that time, intercalator is intercalated into double-strand amplicon as a PCR product. Herein, the intercalator is a fluorescent substance emitting fluorescence when it is intercalated into double-strand DNA, thus intensity of fluorescence emitted from the intercalator is an indicator indicating the amount of amplicon.

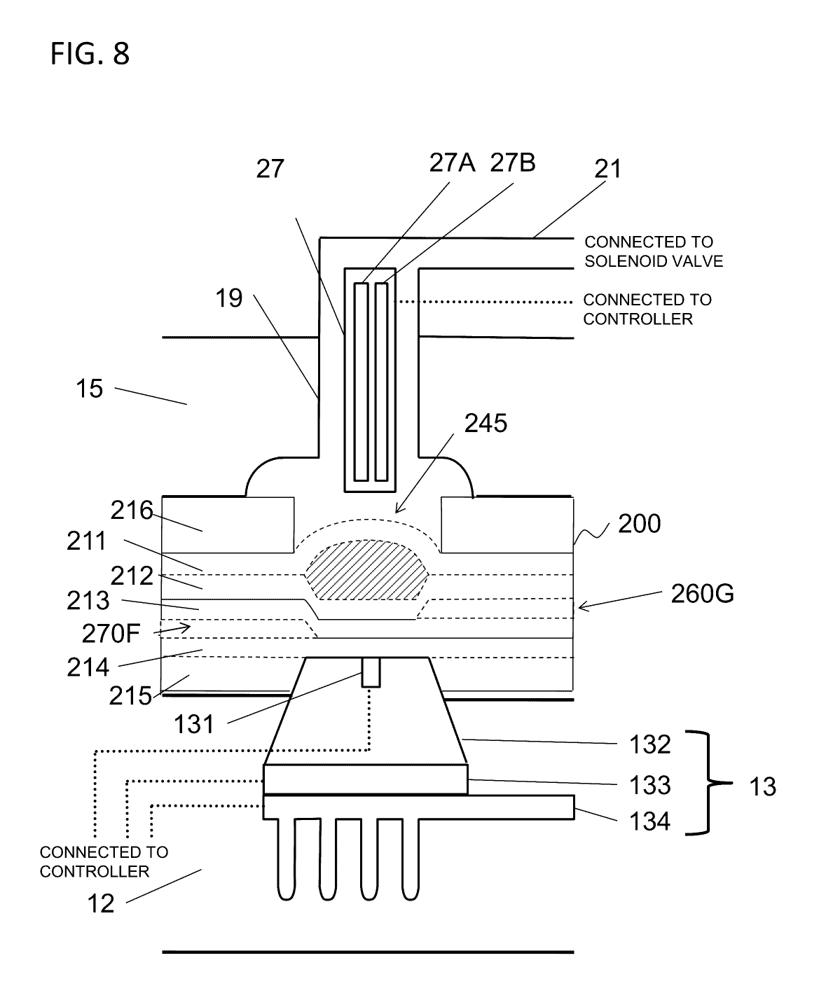

As shown in FIG. 8, a part of the resin plate 215 corresponding to the PCR section 245 is perforated so as to receive temperature control by the temperature control unit 13 via the elastic sheets 212 to 214. The temperature control unit 13 is embedded and arranged in one region on the table 12 and comprises a temperature sensor 131, heat conductor 132, Peltier element 133 and heat releasing plate 134.

The temperature sensor 131 is connected to the controller 24 and measures temperature in the PCR section 245 to send it to the controller 24. One surface of the heat conductor 132 contacts to temperature applying surface of the Peltier element 133 and the other surface of the heat conductor 132 opposing to the Peltier element 133 is exposed from surface of the table 12. The exposed surface of the heat conductor 132 contacts to the microchip 200 so that temperature on the heat conductor 132 is conducted to the PCR section 245 via the elastic sheets 212 to 214.

Power supply line of the Peltier element 133 is connected to the controller 24, and the controller 24 acquires temperature on the PCR section 112 [sic, PCR section 245] from the temperature sensor 131 and determine direction of electric current supplied to the Peltier element 133 based on the acquired temperature so as to carry out temperature control of the Peltier element 133. That is, the Peltier element 133 is a means for heating and cooling sample solution in the PCR section 245.

In addition, as shown in FIG. 8, a pressurizing hole 19 is also arranged on a part corresponding to the PCR section 245, and the pressurizing hole 19 is communicated with the solenoid valve 22 through a tube 21. In addition, an amplicon amount monitoring unit 27 is arranged in a hollow part of the pressurizing hole 19 and tube 21 so as to inject/eject pressurizing medium through outside of the amplicon amount monitoring unit 27.

As shown in FIG. 8, the amplicon amount monitoring unit 27 comprises a light source 27a irradiating excitation light and a receiving part 27b receiving fluorescence, and being connected to the controller 24. The light source 27a is a means irradiating light for exciting fluorescent substance whose intensity is changed together with amplification of amplicon, which comprises, for example, argon ion laser, a filter passing only specific wavelength. The receiving part 27b comprises a photographing element, such as CCD (Charge Coupled Device), and measures intensity in the received light to output the measured value to the controller 24. Herein, the light source 27a and the receiving part 27b are arranged in a manner where laser beam irradiated from the light source 27a and optical axis of the fluorescence received by the receiving part 27b are inconsistent with each other.

Herein, although being omitted in FIG. 8, the amplicon amount monitoring unit 27 is fixed on a plurality of support bars extending from the lid 15, but not floating in inner section of the pressurizing hole 19 and tube 21 on the lid 15. It is preferable that gap between the support bars has a broadness that the injection/ejection of pressurizing medium via the pressurizing holes 19 is never interrupted. In addition, in a case where a control line for controlling the amplicon amount monitoring unit 27 is arranged through the tube 21, it is preferable to reinforce it so that pressurizing medium is not leaked from the through hole.

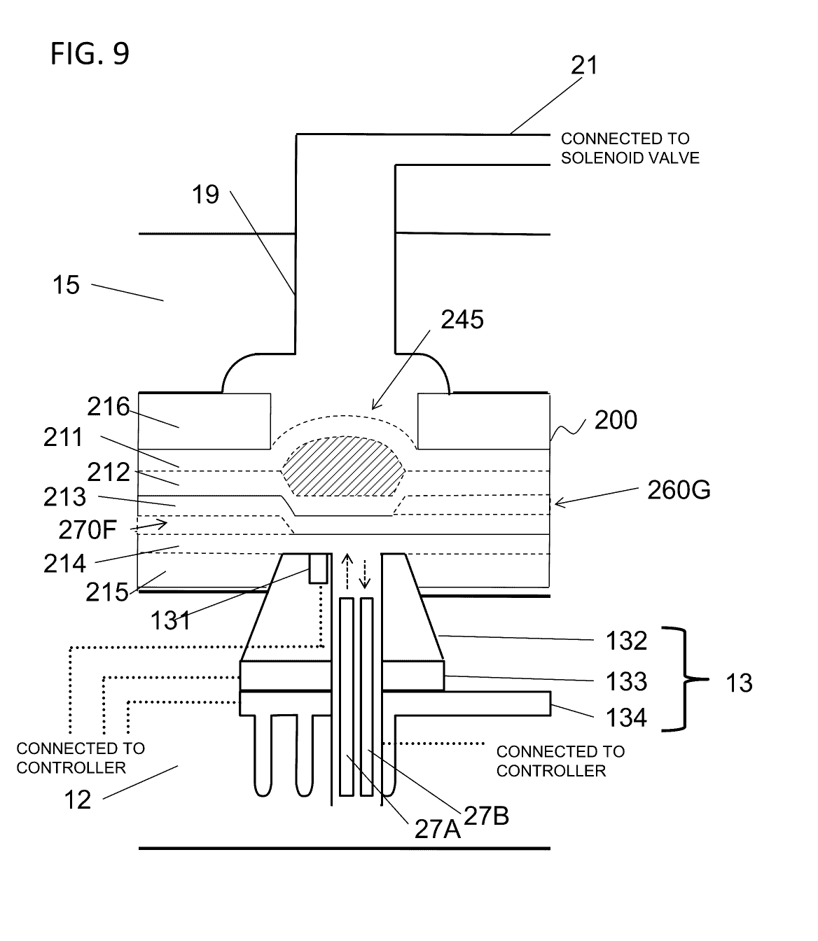

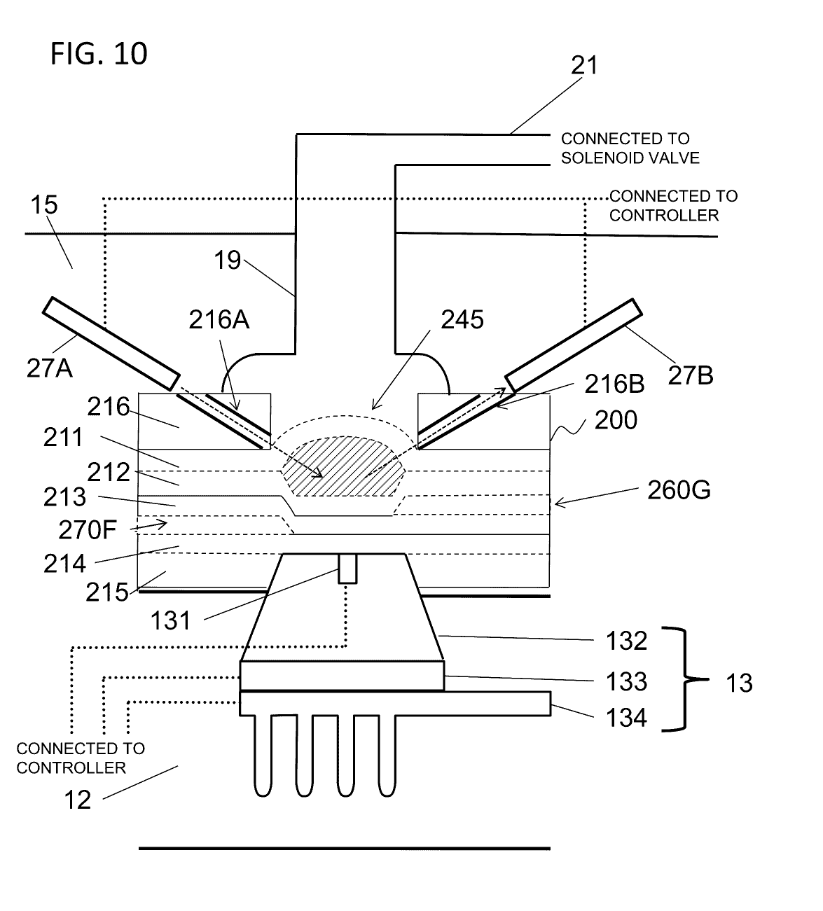

Construction shown in FIG. 8 is a mere exemplification, thus various modification may be applied. For example, as shown in FIG. 9, a hole through the heat conductor 132, Peltier element 133, heat releasing plate 134 of the temperature control unit 13 may be formed and the amplicon amount monitoring unit 27 (the light source 27a, receiving part 27b) may be arrange in inner section of the through hole. Or, as shown in FIG. 10, a construction may be adopted, in which the light source 27a and the receiving part 27b are arranged in inner section of the lid 15 so that laser beam is irradiated in oblique direction onto sample solution in the PCR section 245 and then fluorescence is received. In such case, hole parts 216A, B are formed on the resin plate 216 so that laser beam reaches sample solution in the PCR section 245 in order to ensure optical path. Or, an embodiment may be adopted, in which the light source 27a and the receiving part 27b are arranged above the lid 15 and a part of lid 15 is perforated to ensure an optical path. Anyway, various modifications may be considered in arrangement of the amplicon amount monitoring unit 27, any construction would be adopted if laser beam etc. irradiated from the light source 27a reaches sample solution in the PCR section 245 and fluorescence reaches the receiving part 27b.

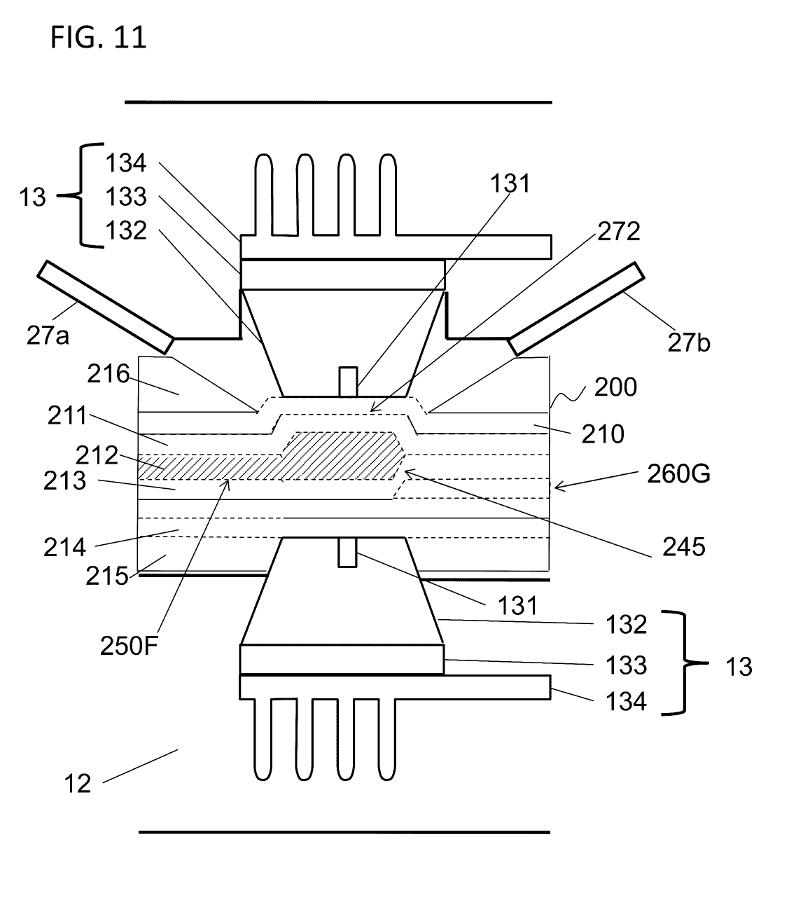

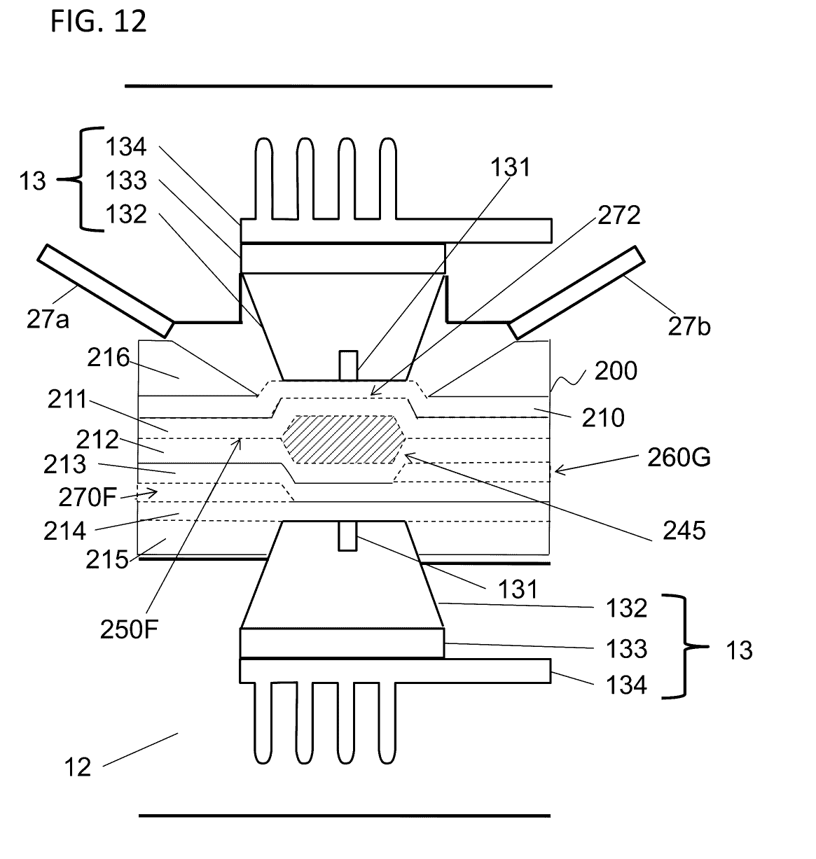

In addition, although constructions has been explained with reference to FIG. 8 to FIG. 10, in which a temperature control unit 13 comprising the Peltier element 133 is arranged below the microchip 200, the temperature control unit 13 may be arranged above the microchip 200 (the side of the lid 15). Or, the temperature control units 13 may be arranged above and below the microchip 200 so as to sandwich it. In such case, as shown in FIG. 11, under a state where flow path 250G as a downstream path communicated to the PCR section 245 is closed and flow path 250F as an upstream path communicated to the PCR section 245 is opened, sample solution is transferred to the PCR section 245 by applying pressurizing medium to the DNA extracting section 244. In addition, as shown in FIG. 12, by injecting pressurizing medium into the flow path opening/closing section 270F, the flow path 250F as a downstream path communicated to the PCR section 245 is closed so that the solution is enclosed in the PCR section 245. Herein, closing of flow path 250F is not essential, for example, a condition in which pressurizing medium is applied to the DNA extracting section 244 may be maintained in order to leave partial solution in the low path 250F.

In addition, in a case where the temperature control units 13 are arranged above and below the microchip 200, for example, fifth elastic sheet 210 is added on first elastic sheet 211 to form a liquid chamber opening/closing part 272 above the PCR section 245, which comprises similar construction with the flow path opening/closing sections 260, 270. Since the PCR section 245 is squashed by injection of pressurizing medium into the liquid chamber opening/closing part 272, solution may be ejected from the PCR section 245. Or, the temperature control unit 13 on at least either of upperside or lowerside is machined to comprise a large number of fine through holes and pressurizing medium is applied from the through holes so that sample solution may be transferred from PCR section 245. Herein, in a case where the fine through holes are arranged on the heat conductor 132, Peltier element 133 and heat releasing plate 134 of the temperature control unit 13, configurations of the heat releasing plate 134 and the like have a construction in which application of pressurizing medium is not disturbed.

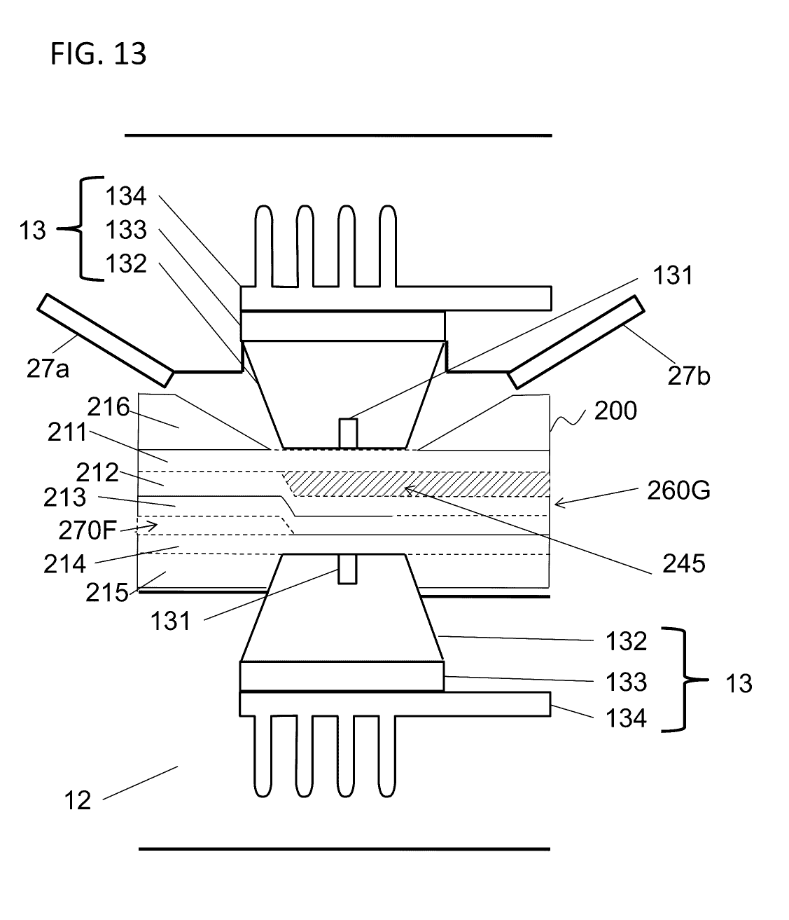

Or, the temperature control unit 13 at the side of the lid 15 is constructed so that it may slide vertically according to control by the controller 24. In such situation, upon carrying out PCR, the temperature control unit 13 at the side of the lid 15 is pressed down to contact to the elastic sheet 211 so that temperature on the heat conductor 132 is conducted to the PCR section 245 via the elastic sheet 211. In addition, upon ejection of sample solution from the PCR section 245, as shown in FIG. 13, the temperature control unit 13 is further pressed down to compress the PCR section 245 so as to realize transfer of sample solution. Herein, in FIG. 11 to FIG. 13, control lines connecting the temperature control unit 13 and amplicon amount monitoring unit 27 with the controller 24 are omitted.

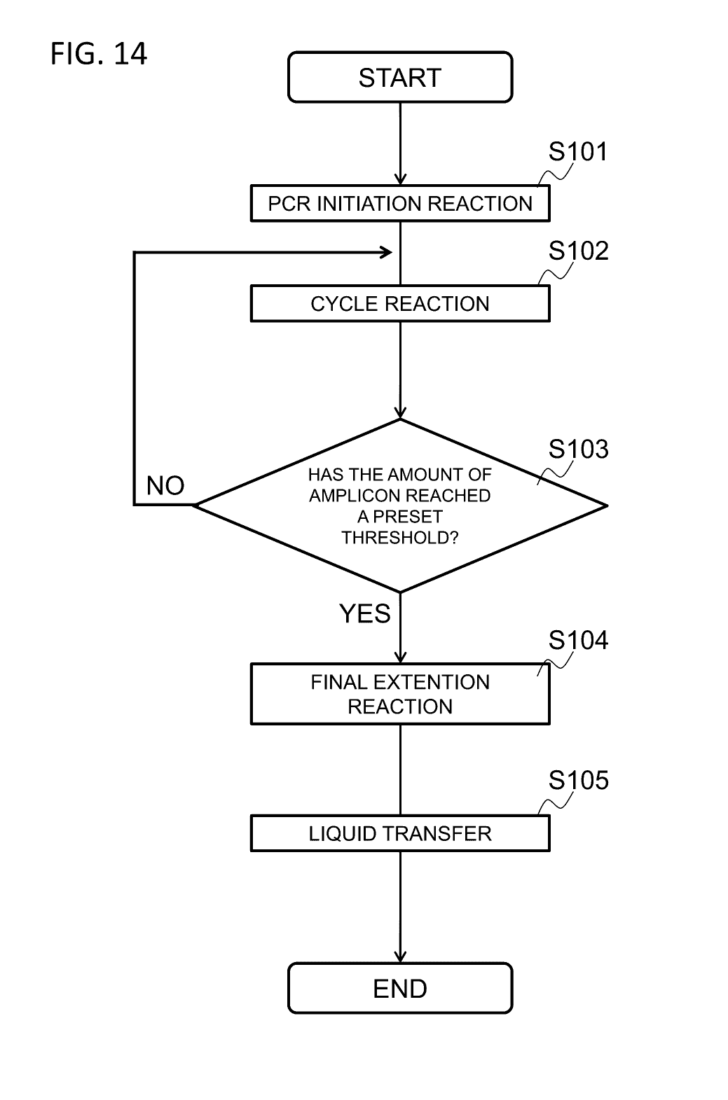

Herein, flow of PCR carried out under control by the controller 24 will be explained. As shown in FIG. 14, after transfer of solution containing sample DNA etc. from the DNA extracting section 244 to the PCR section 245, the controller 24 carries out PCR initiation reaction by controlling the temperature control unit 13 (step S101). The PCR initiation reaction is, for example, hot start process for activating a polymerase.

Then, the controller 24 carries out cycle reaction by controlling the temperature control unit 13 (step S102). The cycle reaction is a reaction in which, for example, a sequential heating and cooling process is repeated, which comprises a step of denaturing reaction for denaturation of double-strand DNA into single-strand DNA, a step of carrying out annealing reaction for hybridization of a primer onto template DNA, and a step of carrying out primer extension reaction with polymerase.

When the sequential heating and cooling process is completed, the controller 24 controls the amplicon amount monitoring unit 27 to measure amount of amplicon, and determines whether the amount of amplicon has reached a preset threshold (step S103). Specifically, the controller 24 instructs the amplicon amount monitoring unit 27 to carry out laser irradiation onto the PCR section 245. The amplicon amount monitoring unit 27 irradiates laser from the light source 27a onto the PCR section 245. In addition, the amplicon amount monitoring unit 27 receives fluorescence emitted from intercalator due to excitation by the laser irradiation, and outputs it as fluorescence intensity to the controller 24. The controller 24 compares the measured value of the fluorescence intensity with a lower allowance threshold registered previously so as to determine whether the amount of amplicon has reached the threshold.

In a case where it is determined that the amount of amplicon is less than the threshold (step S103, branching to NO), the controller 24 controls the temperature control unit 13 to continue the cycle reaction (step S102).

On the other hand, in a case where it is determined that the amount of amplicon is equivalent to threshold or more (step S103, branching to YES), the controller 24 controls the temperature control unit 13 to carry out the final extension reaction (step S104). The final extension reaction is, for example, a reaction for adenylation of the amplicon (maintained at 60° C. for 5 minutes). After that, the controller 24 controls the solenoid valve 22 to transfer partial liquid in PCR section 245 to the volume determination section 246 (step S105) and completes PCR.

Accordingly, the controller 24 carries out: an amplification step in which solution containing sample DNA etc. is heated and cooled so that desired nucleic acid sequence is amplified; a measurement step in which amount of amplicon as amplified nucleic acid sequence is measured; an amplification termination step in which amplification of the desired nucleic acid sequence is terminated based on the measured amount of amplicon. More specifically, the controller 24 carries out determination step in which it is determined whether the measured amount of amplicon has reached a preset threshold, and then, in a case where the measured amount of amplicon has reached the preset threshold, amplification of the desired nucleic acid sequence is terminated.

Herein, PCR condition may be adjusted according to length and nucleic acid sequence of DNA of the purpose of amplification. For example, a primer set is a set of primers for amplifying DNA, that is, for DNA test, thus time for annealing reaction may be adjusted according to TM (melting temperature) value of the primers.

The volume determination section 246 shown in FIG. 5 is a mechanism for measuring solution comprising amplicon. Specifically, the volume determination section 246 is smaller than the PCR section 245, upon liquid transfer from PCR section 245, the microchip controlling apparatus 10 closes flow path 250G under a condition where transfer of solution in the PCR section 245 to the volume determination section 246 has not been accomplished. In other words, the microchip controlling apparatus 10 leaves partial solution in the PCR section 245 so that desired volume of solution comprising amplicon is obtained.

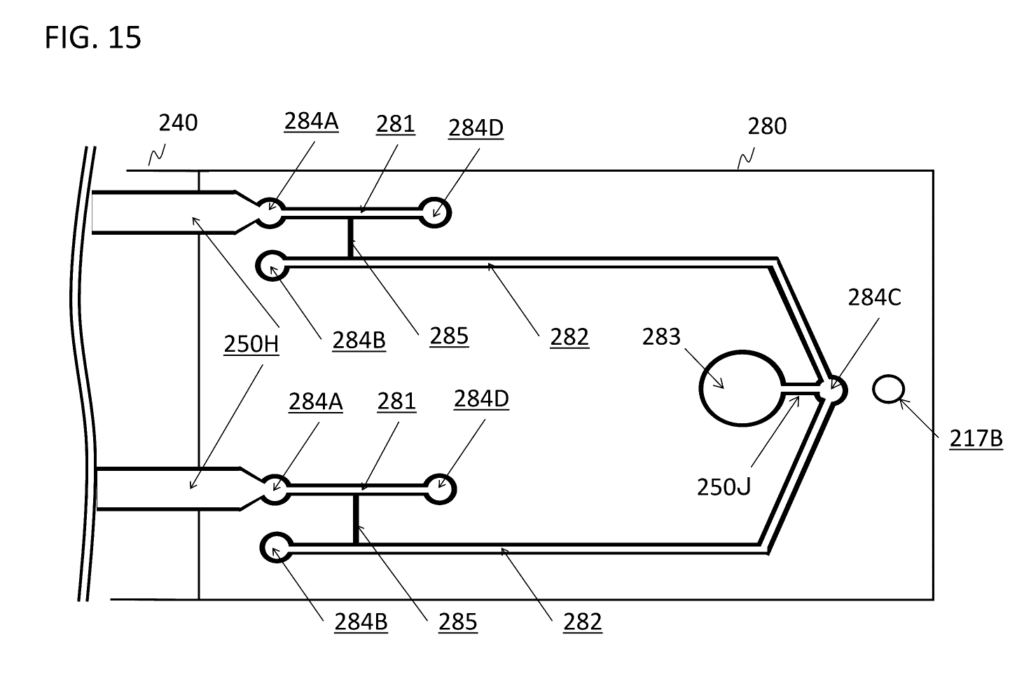

As shown in FIG. 15, the electrophoresis section 280 comprises sample flow paths 281, capillaries 282 and a polymer injection section 283. The microchip controlling apparatus 10 applies electric current to the capillaries 282 via the electrodes 18 so as to carry out electrophoresis, and monitors label flowing through the capillaries with the electrophoresis unit 14 in order to output detection result via a displaying part 28, in which change in fluorescence intensity is graphed in a time dependent manner.

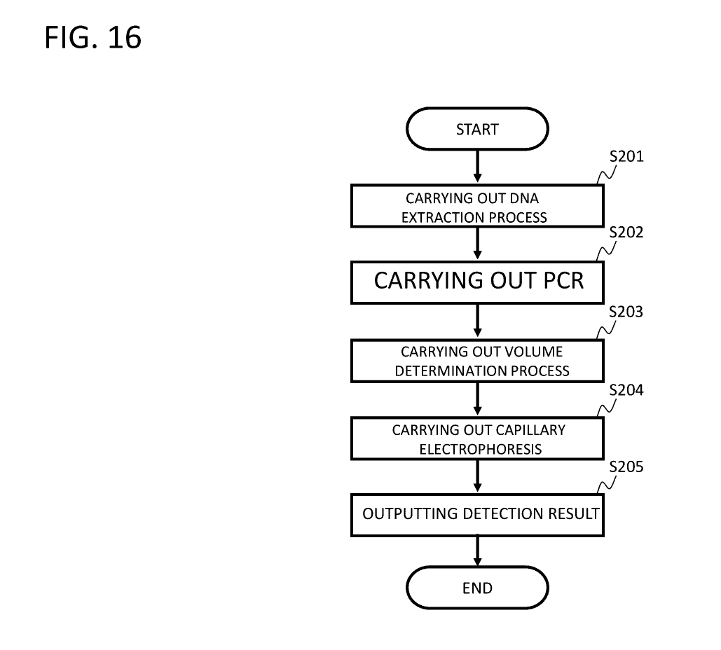

Next, entire flow of DNA analysis process by the microchip controlling apparatus 10 will be explained. Herein, the flow path opening/closing process and the like by the microchip controlling apparatus 10 are omitted for simplifying explanation. In first, a microchip 200 filled up with the sample solution, wash buffer, elution buffer and polymer is set on the microchip controlling apparatus 10 by a user. As shown in FIG. 16, the microchip controlling apparatus 10 carries out DNA extraction process in the DNA extracting unit 25 (step S201).

Then the microchip controlling apparatus 10 carries out PCR (step S202) and volume determination process (step S203). In addition, the microchip controlling apparatus 10 carries out capillary electrophoresis and label detection process (step S204), and then outputs detection result via the displaying part 28 (step S105 [sic, S205]).

Accordingly, upon amplification of amplicon, the microchip controlling apparatus 10 of the first embodiment monitors amount of amplified amplicon at every timing of completion of cycle reaction. As a result, even in sample solution in which DNA amount is unknown, amplicon may be amplified at a suitable amount. For example, when the present apparatus is used for test on humans, in sample solution directly obtained from a donor, in which DNA amount is unknown, amplicon may be amplified to a suitable amount, thus signal having a suitable strength may be obtained upon electrophoresis.

### Second Embodiment

Next, second embodiment will be explained.

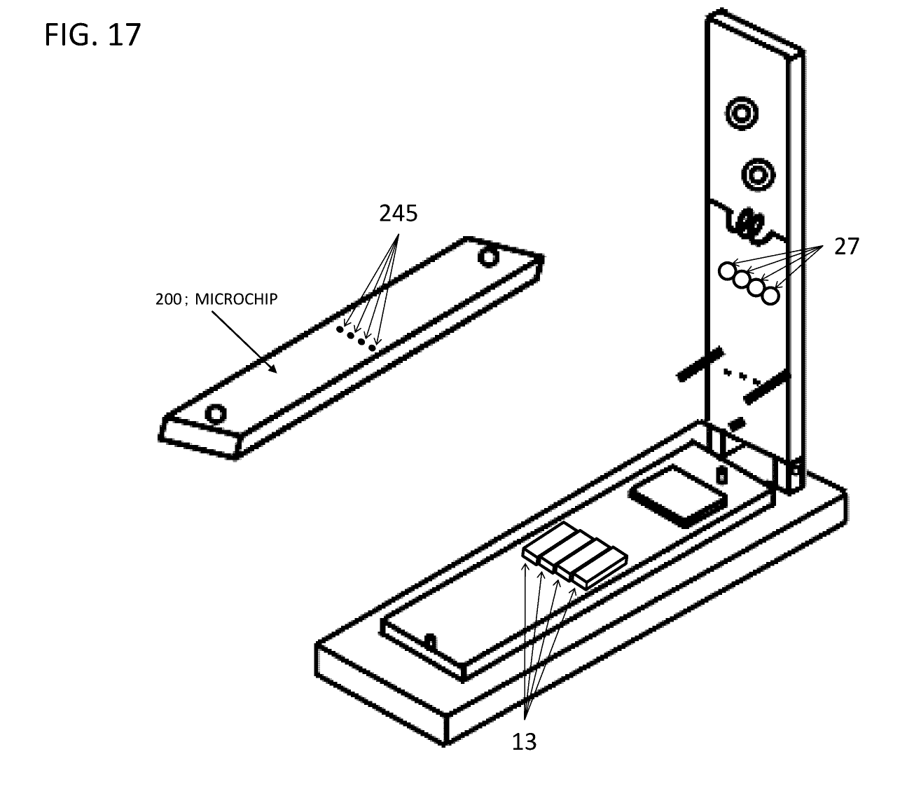

In the second embodiment, as shown in FIG. 17, the microchip 200 comprises a plurality of PCR sections 245, and the microchip controlling apparatus 10 comprises multiple pairs of the temperature control unit 13 and amplicon amount monitoring unit 27 in a manner where each pair respectively corresponds to a PCR section 245. In addition, with respect to a pair of the temperature control unit 13 and the amplicon amount monitoring unit 27 in which the amount of amplicon has reached a preset threshold, the controller 24 carries out final reaction. On the other hand, with respect to a pair of the temperature control unit 13 and the amplicon amount monitoring unit 27 in which the amount of amplicon has not reached the preset threshold, amplification process by the temperature control unit 13 is continued. In addition, when final extension reaction in all reaction paths is terminated, the controller 24 transfers solution in the PCR section 245 to the volume determination section 246.

Thereby, even if there is a difference in amplicon synthesis efficiency between reaction paths, amplicons may be synthesized to a suitable amount. For example, even if there is a difference in amplicon synthesis efficiency between reaction paths due to various reasons, such as difference in primers, amplicon in each of reaction paths may be independently synthesized to a suitable amount.

### Third Embodiment

Next, third embodiment will be explained.

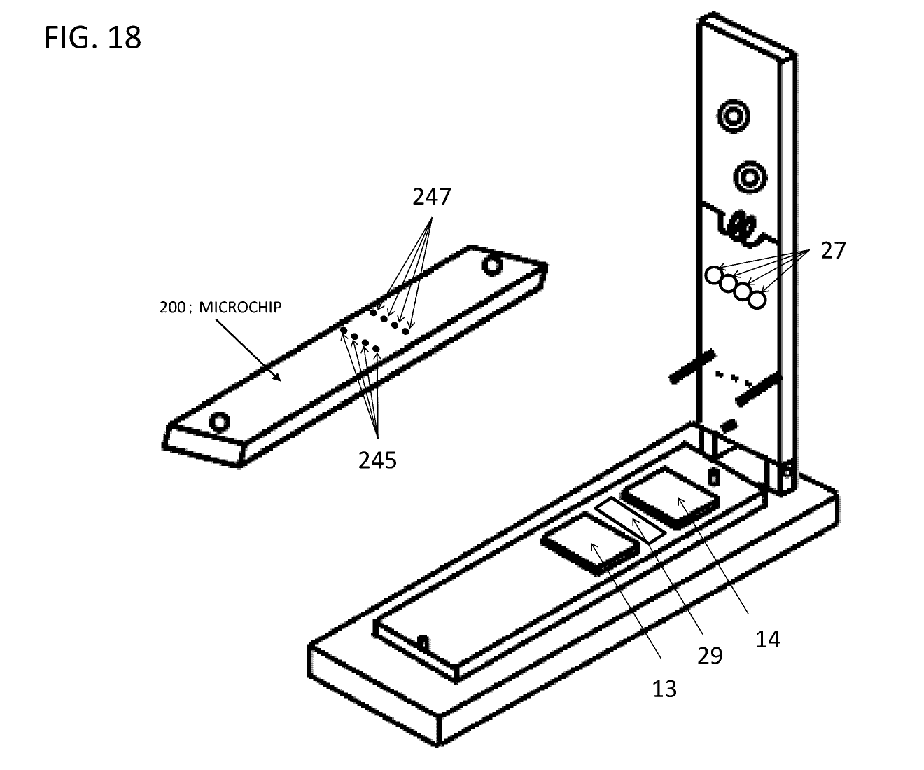

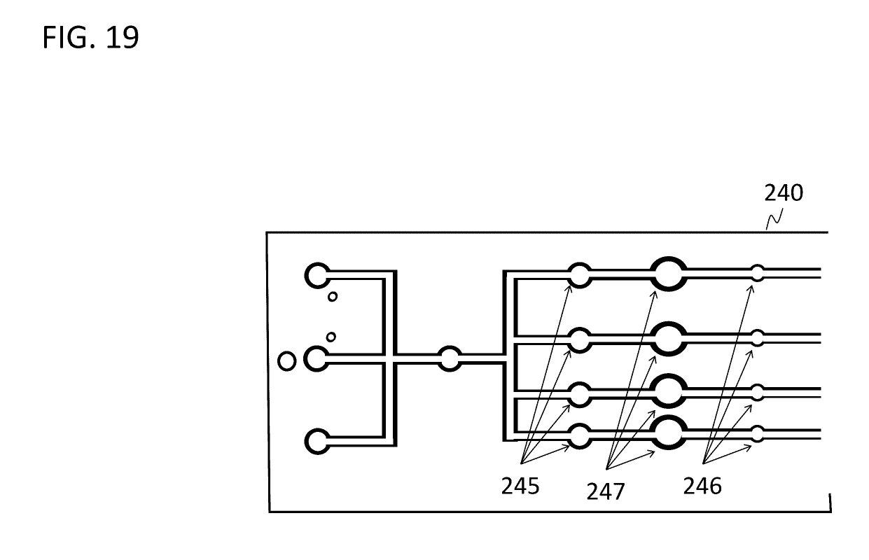

In the third embodiment, as shown in FIG. 18, the microchip 200 comprises a plurality of reaction paths, and comprises the PCR sections 245 and the final reaction section 247 for carrying out the final reaction for each of the reaction paths respectively. Specifically, as shown in FIG. 19, the microchip 200 comprises the final reaction sections 247 between the PCR sections 245 and the volume determination sections 246 for each of the reaction paths respectively. Herein, the reaction path means a single flow path from the flow path 250F, through the PCR section 245 to the sample flow path 281 as described above.

In addition, the microchip controlling apparatus 10 further comprises final reaction units 29 which heats sample solution in the final reaction sections 247 to carry out the final reaction. Specifically, as shown in FIG. 18, the microchip controlling apparatus 10 comprises the final reaction unit 29 between the temperature control unit 13 and the electrophoresis unit 14.

The temperature control unit 13 is so constructed that individual sample solution in the PCR sections 245 is heated and cooled at once in order to carry out amplification reaction on the plurality of reaction path in parallel. In addition, amplicon amount monitoring units 27 are arranged in an associated manner to each PCR section 245 to individually monitor the amount of amplicon in respective PCR section 245. The final reaction unit 29 comprises a heat conductor, Peltier element (thermoelectric element), heat releasing plate etc. and being arranged on the base station 11 like as the temperature control unit 13 to carry out final reaction by, for example, heating sample solution in the final reaction sections 247 at 60° C.

In addition, with respect to a reaction path in which the amount of amplicon has reached the preset threshold, the controller 24 transfers sample solution in the PCR section 245 to the final reaction section 247; and with respect to a reaction path in which the amount of amplicon has not reached the preset threshold, the controller 24 continues the amplification process. The controller 24 repeats a sequential heating/cooling process and measurement of the amplicon amount until amount of amplicon in all of the reaction paths reaches the threshold. After elapsed time from transfer of solution to the final reaction sections 247 in all reaction paths reaches a preset final reaction time, the controller 24 transfers solution in the final reaction sections 247 to the volume determination sections 246.

Accordingly, even if there is a difference in amplicon synthesis efficiency between the reaction paths, the amplicon may be synthesized at a suitable amount.

The other embodiments disclosed in the present application will be explained below. PCR is not limited to that carried on a microchip. For example, the content disclosed in the present application may be applied to PCR carried out at a laboratory etc. That is, the amplicon amount monitoring unit 27 may be installed in a thermal cycler, and programmed to increase/decrease the cycle number according to the amplicon amount.

In addition, sample condition, PCR condition, measurement condition for amplicon amount, electrophoresis condition and the like may be modified variously. For example, sample solution(s) analyzed at once are not limited to sample solution obtained from the same subject, those obtained from a plurality of subjects may be applied. In such case, although amount of template DNA contained in the sample solutions would be different from each other, according to the disclosure in the present application, amplicon may be synthesized at a suitable amount in all sample solutions.

Furthermore, PCR condition may be modified variously according to types of sequence to be amplified, primer, polymerase etc.

In the measurement of the amplicon amount, various technologies relating to RTPCR (Real-time polymerase chain reaction), such as so-called incalation [intercalation] method, TaqMan probe method, and cycling probe method, may be used.

Although double-strand amplicon is subjected to the electrophoresis in the first embodiment, the electrophoresis may be carried out after denaturation into single-strand. For example, a denaturation section is arranged between the PCR section 245 and the volume determination section 246 on the microchip 200. In addition, the microchip controlling apparatus 10 comprises a temperature control unit for temperature control of the denaturation section at, for example, 98° C. Thereby, electrophoresis with single-strand may be realized. Herein, the microchip 200 may be so constructed that denaturing agent, such as formamide, is supplied to the sample solution in the denaturation section.

A part or all of embodiments disclosed above may be described as following modes, but not limited thereto.

### [Mode 1]

The same as the amplification apparatus disclosed in first aspect above.

### [Mode 2]

The amplification apparatus according to Mode 1, wherein the control unit terminates the amplification process by the amplification unit in a case where the amount of amplicon monitored by the monitoring unit reaches a preset threshold.

### [Mode 3]

The amplification apparatus according to Mode 1 or 2, wherein the amplification apparatus comprises multiple pairs of the amplification unit and the monitoring unit, and
with respect to a pair of the amplification unit and the monitoring unit in which the amount of amplicon has reached the preset threshold, the control unit terminates the amplification process by the amplification unit and makes the amplification unit to carry out final reaction in which the amplicon is heated; and with respect to a pair of the amplification unit and the monitoring unit in which the amount of amplicon has not reached the preset threshold, the control unit continues the amplification process by the amplification unit.

### [Mode 4]

The amplification apparatus according to Mode 1 or 2, wherein on a microchip comprising a plurality of reaction paths, and comprising amplification chamber for amplifying the desired nucleic acid sequence and final reaction chamber for carrying out the final reaction for each of the reaction paths respectively,
the amplification unit carries out amplification reaction in the plurality of reaction paths in parallel by heating and cooling the sample solutions in the amplification chambers for the plurality of reaction paths at once, the monitoring unit individually monitors the amount of amplicon in the amplification chamber for each of the plurality of reaction paths,
with respect to the reaction path in which the amount of amplicon has reached the preset threshold, the control unit transfers sample solution in the amplification chamber to the final reaction chamber; and with respect to reaction path in which the amount of amplicon has not reached the preset threshold, the control unit continues amplification process by the amplification unit, and
the amplification apparatus further comprises final reaction units carrying out final reaction by heating sample solution in the final reaction chambers.

### [Mode 5]

The amplification apparatus according to any of Modes 1 to 4, wherein the amplification unit amplifies the desired nucleic acid sequence in a cycle reaction in which a sequential heating and cooling process is repeated, and
the control unit determines whether the amplification process by the amplification unit should be terminated or continued by every completion of a sequential heating and cooling process, based on the amount of amplicon monitored by the monitoring unit.

### [Mode 6]

The amplification apparatus according to any of Modes 1 to 5, wherein the monitoring unit comprises
a light source emitting light exciting fluorescent substance whose intensity is changed together with amplification of amplicon, and
a means receiving fluorescence emitted from the fluorescent substance.

### [Mode 7]

The amplification apparatus according to any of Modes 1 to 6, wherein the amplification unit comprises a thermoelectric element heating and cooling the sample solution and a temperature sensor measuring temperature of the sample solution, and
the control unit performs temperature control on the thermoelectric element based on temperature measured by the temperature sensor.

### [Mode 8]

The same as the amplification method disclosed in second aspect above.

### [Mode 9]

The amplification method according to Mode 8, further comprising a determination step in which it is determined whether the measured amplicon amount has reached a preset threshold; and
in the amplification termination step, amplification of the desired nucleic acid sequence is terminated in a case where the measured amplicon amount has reached the preset threshold

### [Mode 10]

The amplification method according to Mode 9, wherein
in the amplification step, desired nucleic acid sequence is amplified by individually heating and cooling the sample solution divided into a plurality of reaction paths;
in the measurement step, the amplicon amount is measured for each of the reaction paths;
in the determination step, it is determined for each of the reaction paths whether the measured amplicon amount has reached the preset threshold;
in the amplification termination step, with respect to a reaction path in which the amount of amplicon has reached the preset threshold, amplification of the desired nucleic acid sequence is terminated; and
with respect to a reaction path in which the amount of amplicon has not reached the preset threshold, amplification of the desired nucleic acid sequence is continued.

### [Mode 11]

The amplification method according to Mode 9, wherein
in the amplification step, the sample solution divided into the plurality of reaction paths is heated and cooled at once so that desired nucleic acid sequences are amplified;
in the measurement step, the amount of amplicon of each of the reaction paths are measured;
in the determination step, it is determined whether the measured amplicon amount has reached the preset threshold for each of the reaction paths;
in the amplification termination step, with respect to a reaction path in which the amount of amplicon has reached a preset threshold, amplification of the desired nucleic acid sequence is terminated; and
with respect to a reaction path in which the measured amplicon amount has not reached the preset threshold, amplification of the desired nucleic acid sequence is continued.

### [Mode 12]

The same as the amplification system disclosed in third aspect above.

### [Mode 13]

The amplification system according to Mode 12, wherein the control unit terminates the amplification process by the amplification unit in a case where the amount of amplicon monitored by the monitoring unit has reached a preset threshold.

### [Mode 14]

The amplification system according to Mode 12 or 13, wherein
the microchip comprises a plurality of amplification chambers; and
the amplification apparatus comprises multiple pairs of the amplification unit and monitoring unit in a corresponding manner respectively to the amplification chambers;
with respect to a pair of the amplification unit and the monitoring unit in which the amount of amplicon has reached the preset threshold, the control unit terminates the amplification process by the amplification unit and makes the amplification unit to carry out final reaction in which the amplicon is heated; and with respect to a pair of the amplification unit and the monitoring unit in which the amount of amplicon has not reached the preset threshold, the control unit continues the amplification process by the amplification unit.

### [Mode 15]

The amplification system according to Mode 12 or 13, wherein
the microchip comprises a plurality of reaction paths and comprises the amplification chamber and the final reaction chamber for carrying out final reaction for each of the reaction paths;
the amplification apparatus further comprises a final reaction unit for heating sample solution in the final reaction chamber to carry out the final reaction;
the amplification unit carries out amplification reaction in the plurality of reaction paths in parallel by heating and cooling the sample solutions in the amplification chambers for the plurality of reaction paths at once;
the monitoring unit individually monitors the amount of amplicon in the amplification chamber for each of the plurality of reaction paths; and
with respect to a reaction path in which the amount of amplicon has reached a preset threshold, the control unit transfers sample solution in the amplification chamber to a final reaction chamber; and with respect to reaction paths in which the amount of amplicon has not reached to the preset threshold, the control unit continue the amplification process by the amplification unit.

### [Mode 16]

The amplification system according to any of Modes 12 to 15, wherein the amplification unit amplifies the desired nucleic acid sequence in a cycle reaction in which a sequential heating and cooling process is repeated;
the control unit determines whether the amplification process by the amplification unit should be terminated or continued by every completion of a sequential heating and cooling process, based on the amount of amplicon monitored by the monitoring unit

### [Mode 17]

The amplification system according to any of Modes 12 to 16, wherein the monitoring unit comprises:
a light source emitting light exciting fluorescent substance whose intensity is changed together with amplification of amplicon; and
a means receiving fluorescence emitted from the fluorescent substance in the amplification chamber.

### [Mode 18]

The amplification system according to any of Modes 12 to 17, wherein the amplification unit comprises a thermoelectric element heating and cooling the sample solution and a temperature sensor measuring temperature of the sample solution; and
the control unit carries out temperature control on the thermoelectric element based on temperature measured by the temperature sensor.

### [Mode 19]

A program executed by a computer controlling an amplification apparatus comprising:
an amplification means amplifying desired nucleic acid sequence by heating and cooling sample; and
a measurement means measuring amount of amplicon as amplified nucleic acid sequence; wherein the computer executes
a process in which it is determined whether the measured amount of amplicon is a suitable amount or not; and
a process in which, in a case where the measured amount of amplicon is a suitable amount, the amplification by the amplification means is terminated.
Herein, the program may be stored in a storage medium capable of being read by the computer. The storage medium may be a non-transient one, such as a semiconductor memory, hard disk, magnetic recording medium and optical recording medium. That is, the present application disclosed may be realized as a computer program product.

The disclosure of the above mentioned Patent Literature is to be incorporated herein by reference. The exemplary embodiments or Examples may be modified or adjusted within the concept of the entire disclosure of the present invention, including claims, based on the fundamental technical concept of the invention. A variety of combinations or selections of the disclosed elements (elements of claims, Examples and drawings) may be made within the context of the claims of the present invention. That is, the present invention may include a wide variety of changes or corrections that may be made by a skilled person in the art in accordance with the total disclosure including the claims and the drawings as well as the technical concept of the invention. Particularly, it should be understood that any optional numerical figures or sub-ranges contained in the ranges of numerical values set out herein are specifically stated even in the absence of specific statements.

### REFERENCE SIGNS LIST

10 microchip controlling apparatus
11 base station
12 table
13 temperature control unit
14 electrophoresis unit
15 lid
16 hinge
17A, B pins
18 electrodes
19 pressurizing hole
20 O-rings
21 tubes
22 solenoid valve
23 pressure accumulator
24 controller
25 DNA extracting unit
26 power supplying part
27 amplicon amount monitoring unit
28 displaying part
29 final reaction unit
100 microchip
111 (first) elastic sheet
112 (second) elastic sheet
113 (third) elastic sheet
114 (fourth) elastic sheet
115 resin plate
115A, B recessed parts
116 resin plate
116A control holes
117 space part
121 to 123 liquid chambers
131 temperature sensor
132 heat conductor
133 Peltier element
134 heat releasing plate
141 (first) flow path opening/closing section
142 (second) flow path opening/closing section
143 flow path opening/closing section
144 flow path opening/closing section
200 microchip
210 (fifth) elastic sheet
211 (first) elastic sheet
212 (second) elastic sheet
213 (third) elastic sheet
214 (fourth) elastic sheet
215 resin plate
216 resin plate
216A, B hole parts
217A, B pin holes
219 electrode holes
220A, B medium injecting/ejecting holes
240 DNA extraction/PCR section
240A, B liquid chambers
241 sample solution injection section
241A cover film
242 wash buffer injection section
243 elution buffer injection section
244 DNA extracting section
245 PCR section
246 volume determination section
247 final reaction section
248 confluence point
249 branching point
250A to I, X, Y, Z flow paths
260A, C, E, G, Y (on second middle layer) flow path opening/closing sections
261A medium flow path
270B, D, F, H, X, Z (on third middle layer) flow path opening/closing sections
271B medium flow path
272 liquid chamber opening/closing part
280 electrophoresis section
281 sample flow path
282 capillary
283 polymer injection section
290 space part
300 amplification apparatus
301 amplification unit
302 monitoring unit
303 control unit

What is claimed is:

1. An amplification apparatus, comprising:
an amplification unit amplifying desired nucleic acid sequence by heating and cooling sample solution;
a monitoring unit monitoring amount of amplicon as nucleic acid sequence amplified by the amplification unit;
a control unit terminating amplification process by the amplification unit based on the amount of amplicon monitored by the monitoring unit.

2. The amplification apparatus according to claim 1, wherein the control unit terminates the amplification process by the amplification unit in a case where the amount of amplicon monitored by the monitoring unit reaches a preset threshold.

3. The amplification apparatus according to claim 1, wherein
the amplification apparatus comprises multiple pairs of the amplification unit and the monitoring unit, and
with respect to a pair of the amplification unit and the monitoring unit in which the amount of amplicon has reached the preset threshold, the control unit terminates the amplification process by the amplification unit and makes the amplification unit to carry out final reaction in which the amplicon is heated; and with respect to a pair of the amplification unit and the monitoring unit in which the amount of amplicon has not reached the preset threshold, the control unit continues the amplification process by the amplification unit.

4. The amplification apparatus according to claim 1, wherein
on a microchip comprising a plurality of reaction paths, and comprising amplification chamber for amplifying the desired nucleic acid sequence and final reaction chamber for carrying out the final reaction for each of the reaction paths respectively,
the amplification unit carries out amplification reaction in the plurality of reaction paths in parallel by heating and cooling the sample solutions in the amplification chambers for the plurality of reaction paths at once,
the monitoring unit individually monitors the amount of amplicon in the amplification chamber for each of the plurality of reaction paths,
with respect to the reaction path in which the amount of amplicon has reached the preset threshold, the control unit transfers sample solution in the amplification chamber to the final reaction chamber; and with respect to reaction path in which the amount of amplicon has not reached the preset threshold, the control unit continues amplification process by the amplification unit, and
the amplification apparatus further comprises final reaction units carrying out final reaction by heating sample solutions in the final reaction chambers.

5. The amplification apparatus according to any of claim 1, wherein
the amplification unit amplifies the desired nucleic acid sequence in a cycle reaction in which a sequential heating and cooling process is repeated, and
the control unit determines whether the amplification process by the amplification unit should be terminated or continued by every completion of a sequential heating and cooling process, based on the amount of amplicon monitored by the monitoring unit.

6. The amplification apparatus according to any of claim 1, wherein the monitoring unit comprises
a light source emitting light exciting fluorescent substance whose intensity is changed together with amplification of amplicon, and
a means receiving fluorescence emitted from the fluorescent substance.

7. The amplification apparatus according to any of claim 1, wherein
the amplification unit comprises a thermoelectric element heating and cooling the sample solution and a temperature sensor measuring temperature of the sample solution, and
the control unit performs temperature control on the thermoelectric element based on temperature measured by the temperature sensor.

8. An amplification method, comprising:
amplifying desired nucleic acid sequence by heating and cooling sample solution;
measuring the amount of amplicon as amplified nucleic acid sequence;
terminating the amplification of the desired nucleic acid sequence based on the measured amplicon amount.

9. The amplification method according to claim 8, further comprising determining whether the measured amplicon amount has reached a preset threshold; and
upon the termination, amplification of the desired nucleic acid sequence is terminated in a case where the measured amplicon amount has reached the preset threshold.

10. The amplification method according to claim 9, wherein
during the amplification, desired nucleic acid sequence is amplified by individually heating and cooling the sample solution divided into a plurality of reaction paths;
upon the measurement, the amplicon amount is measured for each of the reaction paths;
upon the determination, it is determined for each of the reaction paths whether the measured amplicon amount has reached the preset threshold;
upon the termination, with respect to a reaction path in which the amount of amplicon has reached the preset threshold, amplification of the desired nucleic acid sequence is terminated; and with respect to a reaction path in which the amount of amplicon has not reached the preset threshold, amplification of the desired nucleic acid sequence is continued.

11. The amplification method according to claim 9, wherein
during the amplification, the sample solutions divided into the plurality of reaction paths are heated and cooled at once so that desired nucleic acid sequences are amplified;
upon the measurement, the amount of amplicon of each of the reaction paths is measured;
upon the determination, it is determined whether the measured amplicon amount has reached the preset threshold for each of the reaction paths;
upon the termination, with respect to a reaction path in which the amount of amplicon has reached a preset threshold, amplification of the desired nucleic acid sequence is terminated; and with respect to a reaction path in which the measured amplicon amount has not reached the preset threshold, amplification of the desired nucleic acid sequence is continued.

12. An amplification system, comprising:
a microchip which comprises a plurality of laminated elastic sheets and in which amplification chambers for amplifying desired nucleic acid sequences are constructed at inadhesive site between the elastic sheets; and
an amplification apparatus comprising an amplification unit amplifying desired nucleic acid sequence by heating and cooling sample solutions in the amplification chambers; a monitoring unit monitoring amount of amplicon in the amplification chambers; and a control unit terminating amplification process by the amplification unit based on the amount of amplicon monitored by the monitoring unit.

13. The amplification system according to claim 12, wherein the control unit terminates the amplification process by the amplification unit in a case where the amount of amplicon monitored by the monitoring unit has reached a preset threshold.

14. The amplification system according to claim 12, wherein
the microchip comprises a plurality of amplification chambers; and
the amplification apparatus comprises multiple pairs of the amplification unit and monitoring unit in a corresponding manner respectively to the amplification chambers;
with respect to a pair of the amplification unit and the monitoring unit in which the amount of amplicon has reached the preset threshold, the control unit terminates the amplification process by the amplification unit and makes the amplification unit to carry out final reaction in which the amplicon is heated; and with respect to a pair of the amplification unit and the monitoring unit in which the amount of amplicon has not reached the preset threshold, the control unit continues the amplification process by the amplification unit.

15. The amplification system according to claim 12, wherein
the microchip comprises a plurality of reaction paths and comprises the amplification chamber and the final reaction chamber for carrying out final reaction for each of the reaction paths;
the amplification apparatus further comprises a final reaction unit for heating sample solution in the final reaction chamber to catty out the final reaction;
the amplification unit carries out amplification reaction in the plurality of reaction paths in parallel by heating and cooling the sample solutions in the amplification chambers for the plurality of reaction paths at once;
the monitoring unit individually monitors the amount of amplicon in the amplification chamber for each of the plurality of reaction paths; and
with respect to a reaction path in which the amount of amplicon has reached a preset threshold, the control unit transfers sample solution in the amplification chamber to a final reaction chamber; and with respect to reaction paths in which the amount of amplicon has not reached to the preset threshold, the control unit continue the amplification process by the amplification unit.

16. The amplification system according to claim 12, wherein
the amplification unit amplifies the desired nucleic acid sequence in a cycle reaction in which a sequential heating and cooling process is repeated;
the control unit determines whether the amplification process by the amplification unit should be terminated or continued by every completion of a sequential heating and cooling process, based on the amount of amplicon monitored by the monitoring unit.

17. The amplification system according to claim 12, wherein the monitoring unit comprises:
a light source emitting light exciting fluorescent substance whose intensity is changed together with amplification of amplicon; and
a means receiving fluorescence emitted from the fluorescent substance in the amplification chamber.

18. The amplification system according to claim 12, wherein
the amplification unit comprises a thermoelectric element heating and cooling the sample solution and a temperature sensor measuring temperature of the sample solution; and
the control unit carries out temperature control on the thermoelectric element based on temperature measured by the temperature sensor.
